(19) United States
(12) Patent Application Publication
(10) Pub. No.: US 2023/0137832 A1
(43) Pub. Date: May 4, 2023

(54) PHYSIOLOGY-BASED SUSPICION INDICATOR FOR AN IDENTITY

(71) Applicant: Medtronic, Inc., Mounds View, MN (US)

(72) Inventors: Timothy D. Paffel, Shafer, MN (US); John C. Freeman, Richfield, MN (US); Shinoy B. Cherayil, Plymouth, MN (US)

(21) Appl. No.: 17/960,179

(22) Filed: Oct. 5, 2022

Related U.S. Application Data
(60) Provisional application No. 63/275,476, filed on Nov. 4, 2021.

(51) Int. Cl. A61B 5/117; A61B 5/00; G16H 10/60; G16H 80/00

(52) U.S. Cl. CPC A61B 5/117; A61B 5/7246; G16H 10/60; G16H 80/00

(57) ABSTRACT

Various systems include one or more processors and one or more memory devices storing instructions. The instructions, when executed by the processor(s), cause the system to access temporal physiological data for a person from a storage located remote from the person where the temporal physiological data includes physiological data recorded over time by at least one electronic device of the person, receive a request from a requesting device for a physiology-based suspicion indicator for an identity associated with the person which indicates a degree to which the temporal physiological data of the person reflects a suspicion regarding the identity associated with the person, determine the physiology-based suspicion indicator for the identity based on at least a portion of the temporal physiological data for the person stored remote from the person, and communicate the physiology-based suspicion indicator for the identity associated with the person to the requesting device.

### CROSS-REFERENCE TO RELATED APPLICATIONS

This application claims the benefit of the filing date of and priority to U.S. Provisional Application No. 63/275,476, filed on Nov. 4, 2021, which is hereby incorporated by reference herein in its entirety.

### TECHNICAL FIELD

The present disclosure relates generally to providing a suspicion indicator for an identity, and more particularly, to using temporal physiological data to provide a physiology-based suspicion indicator.

### BACKGROUND

Electronic services are widespread and ubiquitous across the world and include various types of services, such as authentication services, monetary services, legal services, and medical services, among others. Electronic services are generally tied to an identity, which may or may not be checked. Even when an identity is checked in connection with an electronic service, different types of checks involve varying levels of scrutiny. There is continued interest in developing and improving identity checks relating to electronic services.

### SUMMARY

The present disclosure relates to electronic services relating to an identity and relates to providing a physiology-based suspicion indicator for the identity. The electronic services may include authentication services, monetary services, legal services, and medical services, among other services which can be provided electronically. In aspects of the present disclosure, the physiology-based suspicion indicator for an identity is based on temporal physiological data for the person indicated by the identity. As used herein, the term “person” refers to and includes a physical person, and the term “identity” refers to and includes data which specifies, designates, and/or identifies a particular person. The temporal physiological data may include physiological data recorded over time for the person and may be stored remote from the person. The physiology-based suspicion indicator may optionally be based on additional data in addition to the temporal physiological data. The technology of the present disclosure may provide a higher level of scrutiny in identity checks while being less intrusive to the person associated with the identity.

In accordance with aspects of the present disclosure, a system includes one or more processors and one or more memory storing instructions. The instructions, when executed by the one or more processors, cause the system to: access temporal physiological data for a person from a storage located remote from the person where the temporal physiological data includes physiological data recorded over time by at least one physiological device of the person, receive a request from a requesting device for a physiology-based suspicion indicator for an identity associated with the person which indicates a degree to which the temporal physiological data of the person reflects a suspicion regarding the identity associated with the person, determine the physiology-based suspicion indicator for the identity based on at least a portion of the temporal physiological data for the person stored remote from the person, and communicate the physiology-based suspicion indicator for the identity associated with the person to the requesting device.

In various embodiments of the system, the physiology-based suspicion indicator for the identity is determined based on only data gathered prior to the request from the requesting device.

In various embodiments of the system, the physiology-based suspicion indicator for the identity is determined based on only data stored remote from the person associated with the identity.

In various embodiments of the system, the at least one physiological device includes a treatment delivery device, the physiological data recorded over time includes treatment measurements recorded over time by the treatment delivery device, and the physiology-based suspicion indicator for the identity indicates a greater degree of suspicion when the treatment measurements recorded over time indicate non-recommended treatment delivery to the person.

In various embodiments of the system, the temporal physiological data for the person is accessed from among a plurality of stored data for the person. The instructions, when executed by the at least one processor, cause the system to analyze degrees of correlation between the plurality of stored data and a plurality of physiology-based suspicion indicators, and select the temporal physiological data from among the plurality of stored data based on the degrees of correlation.

In various embodiments of the system, the physiology-based suspicion indicator for the identity is determined based on applying at least one of a rules engine or a trained machine learning model to at least the portion of the temporal physiological data for the person.

In various embodiments of the system, the trained machine learning model is trained based on data specific to the person and is customized to the person.

In accordance with aspects of the present disclosure, a processor-implemented method includes accessing temporal physiological data for a person from a storage located remote from the person where the temporal physiological data includes physiological data recorded over time by at least one physiological device of the person, receiving a request from a requesting device for a physiology-based suspicion indicator for an identity associated with the person which indicates a degree to which the temporal physiological data of the person reflects a suspicion regarding the identity associated with the person, determining the physiology-based suspicion indicator for the identity based on at least a portion of the temporal physiological data for the person stored remote from the person, and communicating the physiology-based suspicion indicator for the identity associated with the person to the requesting device.

In various embodiments of the processor-implemented method, determining the physiology-based suspicion indicator for the identity includes determining the physiology-based suspicion indicator for the identity based on only data gathered prior to the request from the requesting device.

In various embodiments of the processor-implemented method, determining the physiology-based suspicion indicator for the identity includes determining the physiology-based suspicion indicator for the identity based on only data stored remote from the person associated with the identity.

In various embodiments of the processor-implemented method, the at least one physiological device includes a treatment delivery device, the physiological data recorded over time includes treatment measurements recorded over time by the treatment delivery device, and the physiology-based suspicion indicator for the identity indicates a greater degree of suspicion when the treatment measurements recorded over time indicate non-recommended treatment delivery to the person.

In various embodiments of the processor-implemented method, the temporal physiological data for the person is accessed from among a plurality of stored data for the person, and the method includes analyzing degrees of correlation between the plurality of stored data and a plurality of physiology-based suspicion indicators, and selecting the temporal physiological data from among the plurality of stored data based on the degrees of correlation.

In various embodiments of the processor-implemented method, determining the physiology-based suspicion indicator for the identity includes determining the physiology-based suspicion indicator for the identity based on applying at least one of a rules engine or a trained machine learning model to the at least the portion of the temporal physiological data for the person.

In various embodiments of the processor-implemented method, the trained machine learning model is trained based on data specific to the person and is customized to the person.

In accordance with aspects of the present disclosure, a non-transitory processor-readable medium stores instructions which, when executed by at least one processor of a system, cause the system to: access temporal physiological data for a person from a storage located remote from the person where the temporal physiological data includes physiological data recorded over time by at least one physiological device of the person, receive a request from a requesting device for a physiology-based suspicion indicator for an identity associated with the person which indicates a degree to which the temporal physiological data of the person reflects a suspicion regarding the identity associated with the person, determine the physiology-based suspicion indicator for the identity based on at least a portion of the temporal physiological data for the person stored remote from the person, and communicate the physiology-based suspicion indicator for the identity associated with the person to the requesting device.

In various embodiments of the non-transitory processor-readable medium, the physiology-based suspicion indicator for the identity is determined based on only data gathered prior to the request from the requesting device.

In various embodiments of the non-transitory processor-readable medium, the physiology-based suspicion indicator for the identity is determined based on only data stored remote from the person associated with the identity.

In various embodiments of the non-transitory processor-readable medium, the at least one physiological device includes a treatment delivery device, the physiological data recorded over time includes treatment measurements recorded over time by the treatment delivery device, and the physiology-based suspicion indicator for the identity indicates a greater degree of suspicion when the treatment measurements recorded over time indicate non-recommended treatment delivery to the person.

In various embodiments of the non-transitory processor-readable medium, the temporal physiological data for the person is accessed from among a plurality of stored data for the person. The instructions, when executed by the at least one processor, further cause the system to analyze degrees of correlation between the plurality of stored data and a plurality of physiology-based suspicion indicators, and select the temporal physiological data from among the plurality of stored data based on the degrees of correlation.

In various embodiments of the non-transitory processor-readable medium, the physiology-based suspicion indicator for the identity is determined based on applying at least one of a rules engine or a trained machine learning model to at least the portion of the temporal physiological data for the person.

In accordance with aspects of the present disclosure, a servicer system includes at least one processor, and one or more memories storing instructions. The instructions, when executed by the at least one processor, cause the servicer system to: receive, from a client device, a request for services relating to an identity associated with a person; communicate, to a physiology integrity system, a request for a physiology-based suspicion indicator for the identity associated the person which indicates a degree to which temporal physiological data of the person reflects a suspicion regarding the identity, where the physiology-based suspicion indicator is based on temporal physiological data which is stored remote from the person and which includes physiological data recorded over time by at least one physiological device of the person; receive, from the physiology integrity system, the physiology-based suspicion indicator for the identity; determine a course of action for the requested services relating to the identity based on the physiology-based suspicion indicator for the identity; and communicate, to the client device, a result of the determination.

In various embodiments of the servicer system, the physiology integrity system which provides the physiology-based suspicion indicator is remote from the client device and from the person.

In various embodiments of the servicer system, the physiology-based suspicion indicator for the identity is determined based on only data gathered prior to the request for the physiology-based suspicion indicator for the identity.

In various embodiments of the servicer system, the physiology-based suspicion indicator for the identity is determined based on only data stored remote from the person.

In various embodiments of the servicer system, the at least one physiological device includes a treatment delivery device, the physiological data recorded over time includes treatment measurements recorded over time by the treatment delivery device, and the physiology-based suspicion indicator for the identity indicates a greater degree of suspicion when the treatment measurements recorded over time indicate non-recommended treatment delivery to the person.

In accordance with aspects of the present disclosure, a processor-implemented method includes: receiving, from a client device, a request for services relating to an identity associated with a person, communicating, to a physiology integrity system, a request for a physiology-based suspicion indicator for the identity associated with the person which indicates a degree to which temporal physiological data of the person reflects a suspicion regarding the identity, where the physiology-based suspicion indicator is based on temporal physiological data which is stored remote from the person and which includes physiological data recorded over time by at least one physiological device of the person; receiving, from the physiology integrity system, the physiology-based suspicion indicator for the identity; determining a course of action for the requested services relating to the identity based on the physiology-based suspicion indicator for the identity; and communicating, to the client device, a result of the determination.

In various embodiments of the processor-implemented method, the physiology integrity system which provides the physiology-based suspicion indicator is remote from the client device and from the person.

In various embodiments of the processor-implemented method, the physiology-based suspicion indicator for the identity is determined based on only data gathered prior to the request for the physiology-based suspicion indicator for the identity.

In various embodiments of the processor-implemented method, the physiology-based suspicion indicator for the identity is determined based on only data stored remote from the person.

In various embodiments of the processor-implemented method, the at least one physiological device includes a treatment delivery device, the physiological data recorded over time includes treatment measurements recorded over time by the treatment delivery device, and the physiology-based suspicion indicator for the identity indicates a greater degree of suspicion when the treatment measurements recorded over time indicate non-recommended treatment delivery to the person.

In accordance with aspects of the present disclosure, a non-transitory processor-readable medium stores instructions which, when executed by at least one processor of a servicer system, cause the servicer system to: receive, from a client device, a request for services relating to an identity associated with a person; communicate, to a system, a request for a physiology-based suspicion indicator for the identity associated with the person which indicates a degree to which temporal physiological data of the person reflects a suspicion regarding the identity, where the physiology-based suspicion indicator is based on temporal physiological data which is stored remote from the person and which includes physiological data recorded over time by at least one physiological device of the person; receive, from the system, the physiology-based suspicion indicator for the identity; determine a course of action for the requested services relating to the identity based on the physiology-based suspicion indicator for the identity; and communicate, to the client device, a result of the determination.

In various embodiments of the non-transitory processor-readable medium, the physiology integrity system which provides the physiology-based suspicion indicator is remote from the client device and from the person.

In various embodiments of the non-transitory processor-readable medium, the physiology-based suspicion indicator for the identity is determined based on only data gathered prior to the request for the physiology-based suspicion indicator for the identity.

In various embodiments of the non-transitory processor-readable medium, the physiology-based suspicion indicator for the identity is determined based on only data stored remote from the person.

In various embodiments of the non-transitory processor-readable medium, the at least one physiological device includes a treatment delivery device, the physiological data recorded over time includes treatment measurements recorded over time by the treatment delivery device, and the physiology-based suspicion indicator for the identity indicates a greater degree of suspicion when the treatment measurements recorded over time indicate non-recommended treatment delivery to the person.

Further details and aspects of exemplary embodiments of the present disclosure are described in more detail below with reference to the appended figures.

### BRIEF DESCRIPTION OF THE DRAWINGS

The above and other aspects and features of the disclosure will become more apparent in view of the following detailed description when taken in conjunction with the accompanying drawings wherein like reference numerals identify similar or identical elements.

FIG. 1 is a diagram of an exemplary networked environment, in accordance with aspects of the present disclosure;

FIG. 2 is a diagram of an exemplary configuration for gathering and conveying physiological data for a person, in accordance with aspects of the present disclosure;

FIG. 3 is a diagram of an exemplary configuration for determining a physiology-based suspicion indicator for an identity, in accordance with aspects of the present disclosure;

FIG. 4 is a diagram of exemplary timelines for the operations FIG. 3, in accordance with aspects of the present disclosure;

FIG. 5 is a diagram of an exemplary operations for using a physiology-based suspicion indicator, in accordance with aspects of the present disclosure;

FIG. 6 is a flow diagram of an exemplary operation of a physiology integrity system, in accordance with aspects of the present disclosure; and

FIG. 7 is a flow diagram of an exemplary operation of a services system, in accordance with aspects of the present disclosure

### DETAILED DESCRIPTION

The present disclosure relates to electronic services relating to an identity and to providing a physiology-based suspicion indicator for the identity. The electronic services may include authentication services (e.g., logging into a system), monetary services (e.g., credit card payment), legal services (e.g., electronically signing an agreement), and medical services (e.g., ordering prescription medication), among other services which can be provided electronically. In aspects of the present disclosure, the physiology-based suspicion indicator for an identity is based on temporal physiological data for the person associated with the identity. The temporal physiological data may include physiological data recorded over time for the person and may be stored remote from the person. In aspects, the physiology-based suspicion indicator may optionally be based on additional data in addition to the temporal physiological data. As mentioned above, the term “person” refers to and includes a physical person, and the term “identity” refers to and includes data which specifies, designates, and/or identifies a particular person. As used herein, the term “remote” refers to and includes a location that is different from the location of a person, including, without limitation, different locations within a building. The technology of the present disclosure may provide a higher level of scrutiny in identity checks while being less intrusive to the person associated with the identity.

Although the disclosure is not limited in this regard, discussions utilizing terms such as, for example, “processing,” “computing,” “calculating,” “determining,” “establishing,” “analyzing,” “checking,” or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other non-transitory information storage media that may store instructions to perform operations and/or processes. As used herein, “exemplary” does not necessarily mean “preferred” and may simply refer to an example unless the context clearly indicates otherwise. Although the disclosure is not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items. Unless explicitly stated, the methods described herein are not constrained to a particular order or sequence. Additionally, some of the described methods or elements thereof can occur or be performed simultaneously, at the same point in time, or concurrently.

Various embodiments of the present disclosure relate to the use of physiological data associated with medical treatment of a patient that is associated with an identity used in connection with an online system or service, such as an online banking service, a social media service, or an email service. While not necessarily limited thereto, various features discussed herein can be readily applicable to such embodiments.

According to various embodiments of the present disclosure, patient information for medical treatment purposes can include longitudinal patient records that span days, months, or even years. The patient information can also include information originating from medical devices, such as implantable cardiac stimulators, insulin pumps, and neurostimulators, among other things. Patient information can be particularly difficult to falsify or spoof for the purposes of impersonating the online identity of an individual. Moreover, the accessibility of such information can be subject to heighten governmental regulations, which can make such information unavailable for general use. Aspects of the present disclosure are directed to a suspicion indicator system that segregates patient information related to the treatment of patients from an interface that is accessible to external computer systems. The external computer systems can send a request to the suspicion indicator system when the identity of an individual attempting to access or utilize the external computer system is in question. This might occur, for example, when the external computer system receives a login request or a request to implement an action that triggers heightened risk (such as changing account information or initiating a financial transaction). Upon receipt of such a request, the suspicion indicator system correlates the request to a patient. The correlation to the patient allows the suspicion indicator system to identify and access the appropriate patient data and associated suspicion-based analysis. The suspicion indicator system uses the analysis to create a suspicion indicator that provides an indication of the level of confidence that the patient is the same as the individual attempting to access the external computer system. The suspicion indicator system can then provide the suspicion indicator to the requesting system.

As one, non-limiting example, a computer system of financial institution may receive a login request corresponding to an online identity for a particular individual. In addition to password or similar protections, the financial computer system can send a request to the suspicion indicator system. The request can include an identifier of the individual; however, the individual need not be directly identifiable from the information request. For instance, the systems can use arbitrary identifiers. The systems can share associations between the arbitrary identifiers to individuals that are known only to the systems such that access to the request does not allow a nefarious party to identify the individual. It is contemplated that other obfuscation and security measures can be taken to protect the identity of the individual. The suspicion indicator system uses the information in the request to identify the relevant patient information, which can then be used to create a corresponding suspicion indicator. The financial computer system can use the suspicion identifier in any number of different ways. For example, if the suspicion identifier indicates a high level of suspicion, the financial computer system could trigger additional security measures such as multifactor identification, CAPTCHA, or temporary account suspension, among other things.

According to various embodiments, the suspicion indicator is be provided back to the financial computer system without providing any patient health information. For example, the suspicion indicator can be representative of a confidence level without any details as to the patient-specific information that went into the determination of the confidence level. This separation between the analysis and the communicated information can be particularly useful for maintaining the security of patient information.

According to various embodiments, the longitudinal patient data includes temporally disparate physiological or other information about the patient. The temporal nature can represent data collected over the course of treatment or monitoring of the patient. For instance, the longitudinal patient data can include information related to an implantable medical device, such as patient treatments administered, monitored patient data, or access history through a patient or clinician programmer. This type of information can then be used by the suspicion indicator system to identify abnormal circumstances related to a particular request from another system. For instance, the patient information may be indicative of a temporal situation where the patient is highly unlikely to be accessing an online system, such as when the patient is undergoing medical treatment inconsistent with the timing of the online access attempts or when the patient is more likely to be asleep. The patient information could also include recent data from medical devices that are indicative of patient status, such as patient activity.

According to various embodiments, the suspicion indicator system can be configured to create the suspicion indicator from independencies between multiple pieces of patient information. For instance, the patient login history to a home health application (e.g., a patient programmer for an implanted medical device) could be correlated with the timing of requests for suspicion indicators, such as where the login history establishes a pattern of similar login attempts for the home health application and other systems that request suspicion indicators (e.g., a financial institution or social networking platform). Machine learning can be used to develop a set of complex interdependencies between multiple variables as relevant to creating a suspicion indicator for a particular request. These interdependencies can be created on a macro (across all individuals, or certain groups if individuals) level, an individual level, or both macro and individual levels.

Consistent with various embodiments of the present disclosure, interdependencies created on the individual level can be utilized to create a specific digital profile. The suspicion indicator system can then use each digital profile to identify anomalies in recent patient data that may result in an elevated suspicion level.

According to various embodiments of the present disclosure, patient data for medical treatment purposes can include physiological data that is collected from physiological devices, such as implantable medical devices. It is understood that patient data other than physiological data can be used in the generation of a suspicion indicator. For ease of discussion, various embodiments are discussed in the context of physiological-based data and physiological devices, without necessarily precluding the use of other patient data that has been collected or maintained as part of medical treatment, whether alone or in addition to the physiological-based data.

According to various embodiments, the suspicion indicator provided by the suspicion indicator system can include information that indicates when the system is not able to formulate a meaningful assessment of the suspicion level. As an example, this situation might occur when the patient in question has little patient health data or when the patient health data. The requesting external system can then utilize this additional information when determining how to proceed with the activity that caused the external system to issue the request.

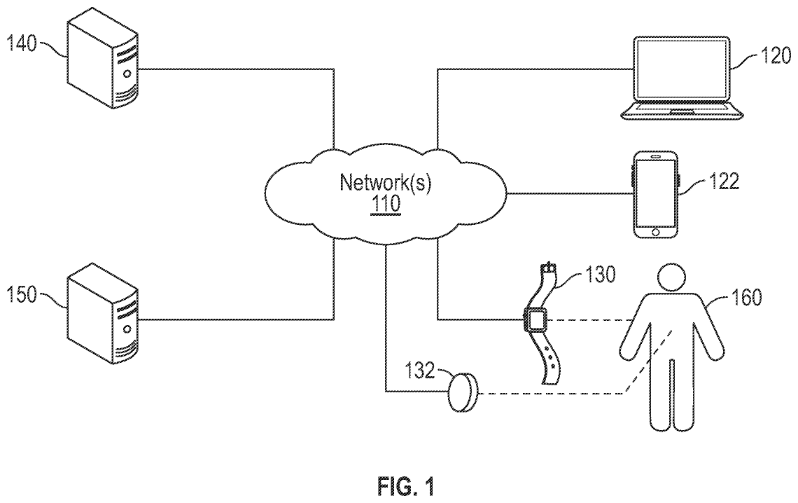

Referring to FIG. 1, there is shown a diagram of an exemplary network environment. The network environment includes one or more communications networks 110 and various devices in communication with the communications network(s) 110. The communications network(s) 110 may include one or more of (or portions of one or more of) wireless networks (e.g., Wi-Fi, Bluetooth, etc.), wired networks (e.g., Ethernet, MoCA, DSL, etc.), cellular networks, satellite networks, local area networks (LAN), wide area networks (WAN), intranets, extranets, virtual private networks (VPN), the Internet, and/or Internet service provider (ISP) networks, among others, and may include various devices and/or components, such as modems, routers, and/or cables, among other things. The devices in communication with the communications network(s) may include user devices/client devices 120, 122, physiological devices 130, 132 which gather physiological data of a person 160, one or more systems 140 which provide services to the user devices, and one or more systems 150 which store physiological data used for a physiology-based suspicion indicator. These are described in more detail below. In the description herein, the terms “user device” and “client device” may be used interchangeably.

The user devices 120, 122 may be any electronic device which can use electronic services of a system 140 which provides services to the user devices 120, 122. The systems 140 may be referred to herein as a “services systems.” In various embodiments, the user devices 120, 122 may include laptops, desktops, tablets, smartphones, smartwatches, dedicated devices for particular services (e.g., Ring® doorbell, automatic teller machine, etc.), dedicated devices for a particular person (e.g., insulin pump, pacemaker, etc.), and/or another type of electronic device. Two types of user devices are shown in FIG. 1 to provide an illustrative example, but any type or number of user devices which can use electronic services may be used.

The user devices 120, 122 communicate with one or more services system(s) 140, which provide services to the user devices 120, 122. The services system(s) 140 may provide any type of service, such as, without limitation, data access (e.g., e-mail access), data storage, product ordering, appointment booking, monetary services, electronic signature services, software as a service (SaaS), software as a medical device (SaMD), and/or other types of services. The services system(s) 140 may provide the services for any type of entity, such as a bank, a merchant, a medical practice, a hospital, a security company, an information technology (IT) department, an electronic signature services company, and/or a professional services company, among others. The services system(s) 140 may include proprietary servers, cloud-based servers, and/or databases, among other things, and persons skilled in the art will understand how to implement such servers to provide the services.

In accordance with aspects of the present disclosure, the services system(s) 140 may provide services which are tied to particular identities of persons, such as, without limitation, personal e-mail services, personal banking services, appointment booking, and/or electronic signature services, among other services tied to particular identities. The services system(s) 140 may require the user devices 120, 122 to indicate the identity for which services are requested. The services system(s) 140 and the user devices may implement the indication of identities in various ways. For example, an identity may be indicated using personal information (e.g., name, birthday, etc.), an e-mail address, a number (e.g., social security number, driver's license number, telephone number, etc.), a username, and/or an alphanumeric identifier, among other things. Persons skilled in the art will understand how to implement such identity indications and to implement communication of such identity indications between the user devices 120, 122 and the services system(s) 140. In accordance with aspects of the present disclosure, the services system(s) 140 may or may not require identity verification, such as passwords, personal knowledge (e.g., answer to particular questions), two-factor authentication, and/or biometric scans (e.g., fingerprint), among other things. Accordingly, in accordance with aspects of the present disclosure, the technology of the present disclosure may be applied so long as an identity is indicated, even when there is no identity verification.

Accordingly, described above are interactions between user devices 120, 122 and services system(s) 140 which provide services to the user devices 120, 122. The following describes interactions between the physiological devices 130, 132 and the system(s) 150 which store physiological data used for a physiology-based suspicion indicator. The system 150 may be referred to herein as a “physiology integrity system.”

The physiological devices 130, 132 may be any device which can gather data relating to the physiological condition of a person. In various embodiments, the physiological devices 130, 132 may include, without limitation, sensing devices which sense a physiological characteristic of a person (e.g., heart rate, blood pressure, temperature, blood glucose, etc.), delivery devices which deliver a substance to a person (e.g., insulin delivery device, albuterol delivery device, etc.), devices which stimulate a portion of a person's body (e.g., pacemaker, etc.), wearable devices (e.g., sensing smart watch, etc.), implanted devices, and/or carried devices, among other things. Two types of physiological devices are shown in FIG. 1 to provide an illustrative example, including a wearable device 130 and an implanted device 132 of a person 160, but any type or number of physiological devices which can gather data relating to the physiological condition of a person may be used.

In accordance with aspects of the present disclosure, the physiological devices 130, 132 of a person 160 may gather physiological data over time, such as automatically gathering physiological data at preset time intervals and/or gathering physiological data over time when triggered to do so (e.g., manual trigger by a person, trigger by a sensor, etc.). Physiological data gathered over time may be referred to herein as “temporal physiological data.” Aspects of the present disclosure below may be described using temporal physiological data as an example. However, it is intended that physiological data in general may be used by the technology of the present disclosure, and such embodiments are contemplated to be within the scope of the present disclosure.

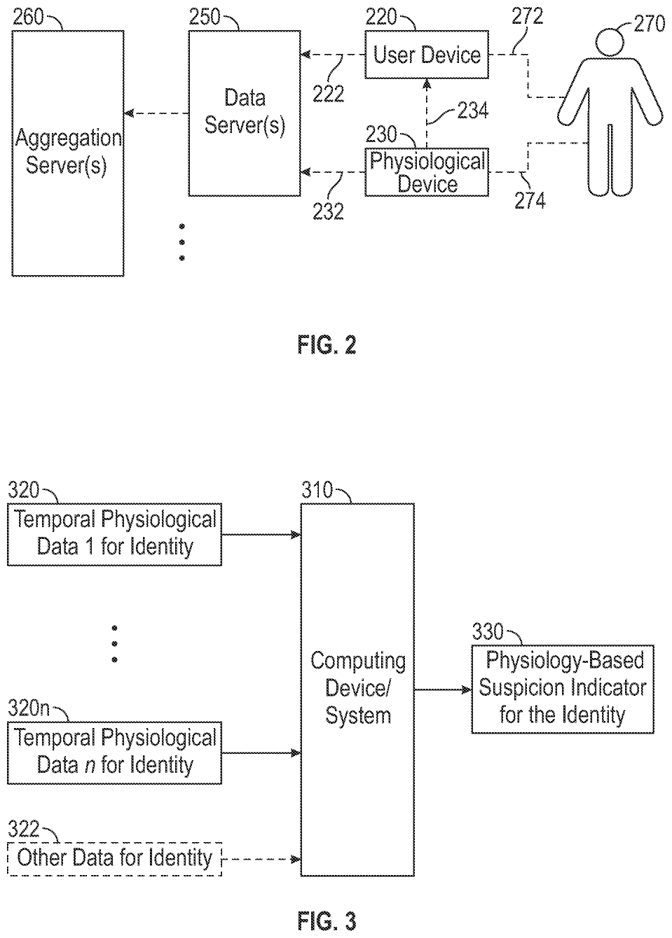

In accordance with aspects of the present disclosure, temporal physiological data gathered by the physiological devices 130, 132 can be conveyed to the physiology integrity system(s) 150 for storage therein. The physiological data is conveyed together with identity information which identifies the person 160 from whom the physiological data was gathered. Various manners of conveying such information, and examples of information stored by the physiology integrity system(s) 150, will be described in more detail in connection with FIG. 2. The physiology integrity system(s) 150 may include proprietary servers, cloud-based servers, and/or databases, among other things. In various embodiments, the physiology integrity system(s) 150 may be servers owned or leased by the manufacturer(s) of the physiological devices 130, 132. In various embodiments, the physiology integrity system(s) 150 may be servers which aggregate physiological data from one or more other data sources. Such and other embodiments are contemplated to be within the scope of the present disclosure.

In accordance with aspects of the present disclosure, the physiological data stored in the physiology integrity system(s) 150 may be used to generate a physiology-based suspicion indicator for the identity associated with the physiological data. Examples of a physiology-based suspicion indicator will be described later herein in connection with FIG. 3. In accordance with aspects of the present disclosure, the services system(s) 140 may request a physiology-based suspicion indicator for a particular identity from the physiology integrity system(s) 150 when the services system(s) 140 is requested to provide services for the particular identity to user devices 120, 122. Based on the indication provided by the physiology-based suspicion indicator, the services system(s) 140 may decide to provide the requested services or may decline to provide the requested services to the user devices 120, 122 or may take other actions based on the indication provided by the physiology-based suspicion indicator. Interactions between the services system(s) 140 and the physiology integrity system(s) 150 will be described in more detail in connection with FIG. 5.

The examples shown and described in connection with FIG. 1 are illustrative, and variations are contemplated to be within the scope of the present disclosure.

Referring now to FIG. 2, there is shown a diagram of an exemplary configuration for conveying physiological data gathered by a physiological device to one or more systems. As described below, communications occur through one or more networks, such as the network 110 described in connection with FIG. 1, but networks are not shown in FIG. 2 to provide clearer illustration. In the illustration of FIG. 2, physiological data may be gathered by a physiological device 230 of a person 270, and the physiological data may be conveyed to one or more data servers 250. The physiological device 230 may be a physiological device as described in connection with FIG. 1, such as a sensing device which sense a physiological characteristic of a person 270, a delivery device which delivers a substance to a person 270, a device which stimulates a portion of a person's body 270, a wearable device, an implanted device, and/or a carried device, among other things. A dashed line 274 is shown in FIG. 2 to indicate that the physiological device 230 performs one or more such functions with respect to the person 270. In various embodiments, the physiological device 230 may be a communications-enabled device which permits the physiological device 230 to communicate with the data server(s) 250 through a physiological device communications link 232. For example, the physiological device 230 may have wired communication capability (e.g., Ethernet, etc.) and/or wireless communication capability (e.g., Wi-Fi, etc.) which enable the physiological device 230 to communicate via the physiological device communications link 232. As mentioned above, the physiological device communications link 232 may span one or more networks (not shown). Using the physiological device communications link 232, the physiological device 230 may communicate physiological data to the data server(s) 250. In various embodiments, the data server(s) 250 and the physiological device 230 (and optionally the user device 220) may be part of a medical treatment platform provided by a medical company, a medical facility, a medical practice, and/or a healthcare provider.

In various embodiments, the physiological device 230 may not communicate with the data server(s) 250. Rather, the physiological device 230 may form a local connection 234 with a user device 220 of the person 270, such as a Bluetooth connection or a USB connection, among other things. Using the local connection 234, the physiological device 230 may convey the physiological data to the user device 220. In turn, the user device 220 may communicate with the data server(s) 250 over a user device communications link 222. For example, the user device 220 may have wired communication capability (e.g., Ethernet, etc.) and/or wireless communication capability (e.g., Wi-Fi, etc.) which enable the user device 220 to communicate via the user device communications link 232. The person 270 may configure the user device 220 to communicate with the physiological device 230 and the data server(s) 250 by installing and using an app or software application in the user device 220, such as an app or software application provided by a manufacturer of the physiological device 230 and/or provided by a treatment service, among other possibilities. As mentioned above, the user device communications link 222 may span one or more networks (not shown). Using the user device communications link 222, the user device 220 may communicate the physiological data, received from the physiological device 230, to the data server(s) 250.

In various embodiments, the physiological device may not form a local connection 234 with the user device 220. Rather, the physiological device may display, speak, or otherwise present the physiological data it gathered (e.g., temperature), and a person may read, hear, or otherwise perceive the physiological data and manually enter it into the user device 220. The user device 220 may then communicate the physiological data to the data server(s) 250 via the user device communications link 222.

Accordingly, as described above, the data server(s) 250 may receive physiological data from the physiological device 230 through the physiological device communications link 232 and/or the user device communications link 222. In accordance with aspects of the present disclosure, the data server(s) 250 store the physiological data together with an identity indicator which identifies an identity of the person from whom the physiological data was gathered. As mentioned above, the identity indicator may include personal information (e.g., name, birthday, etc.), an e-mail address, a number (e.g., social security number, driver's license number, telephone number, etc.), a username, and/or an alphanumeric identifier (e.g., generated by an algorithm, such as a hash function), among other things. In various embodiments, the physiological device 230 may communicate the identity indicator together with the physiological data to the data server(s) 250. In various embodiments, the identity indicator may not be communicated from physiological device 230. Rather, the physiological device 230 may communicate another identifier to the data server(s) 250, such as a physiological device identifier, and the data server(s) 250 may reference a lookup table or other predetermined association to look up the identity associated with the received identifier. Such and other embodiments are contemplated to be within the scope of the present disclosure.

In various embodiments, different data servers may store physiological data for different identities. For example, different data servers may store physiological data for different geographical areas. In various embodiments, a person's physiological data may be stored across different data servers. For example, a person may use multiple physiological devices from different manufacturers, and the physiological devices may store their corresponding physiological data on different data servers. In accordance with aspects of the present disclosure, physiological data across different data servers may be aggregated by one or more aggregation servers 260. In the illustrative example of FIG. 2, the data server(s) 250 and/or the aggregation server(s) 260 may be part of the physiology integrity system(s) 150 of FIG. 1.

The examples shown and described in connection with FIG. 2 are illustrative, and variations are contemplated to be within the scope of the present disclosure.

Referring now to FIG. 3, there is shown a diagram of an exemplary configuration for generating a physiology-based suspicion indicator based on temporal physiological data. In the illustrated embodiment, a computing device or system 310 may be part of the physiology integrity system(s) 150 of FIG. 1 and may be the data server(s) 250 and/or aggregation server(s) 260 of FIG. 2. In various embodiments, the computing device or system 310 may be the user device 220 of FIG. 2, such as a smartphone, smartwatch, or another device local to the person 270.

In accordance with aspects of the present disclosure, the computing device/system 310 may access the temporal physiological data 320-320n for an identity. As mentioned above, the temporal physiological data for an identity includes physiological data gathered and stored over time for the identity. The temporal physiological data may include a number n of temporal physiological data, where n≥1. FIG. 3 illustrates the case where n>1, but n may be equal to 1 as well. Each temporal physiological data may be data gathered by a corresponding physiological device, such as a physiological device as described in connection with FIG. 1. For example, temporal physiological data 1 (320) may be data gathered by a continuous glucose monitor, and physiological data n (320n) may be data gathered by a thermometer, among other things.

The computing device/system 310 may process the temporal physiological data for an identity 320-320n to generate a physiology-based suspicion indicator for the identity 330. In accordance with aspects of the present disclosure, the physiology-based suspicion indicator 330 reflects whether or not the temporal physiological data 320-320n indicates a suspicion regarding the identity and/or may reflect a degree of suspicion regarding the identity. The criteria for a suspicion regarding the identity may vary, and the computing device/system 310 may implement different criteria using a rules engine and/or a machine learning model.

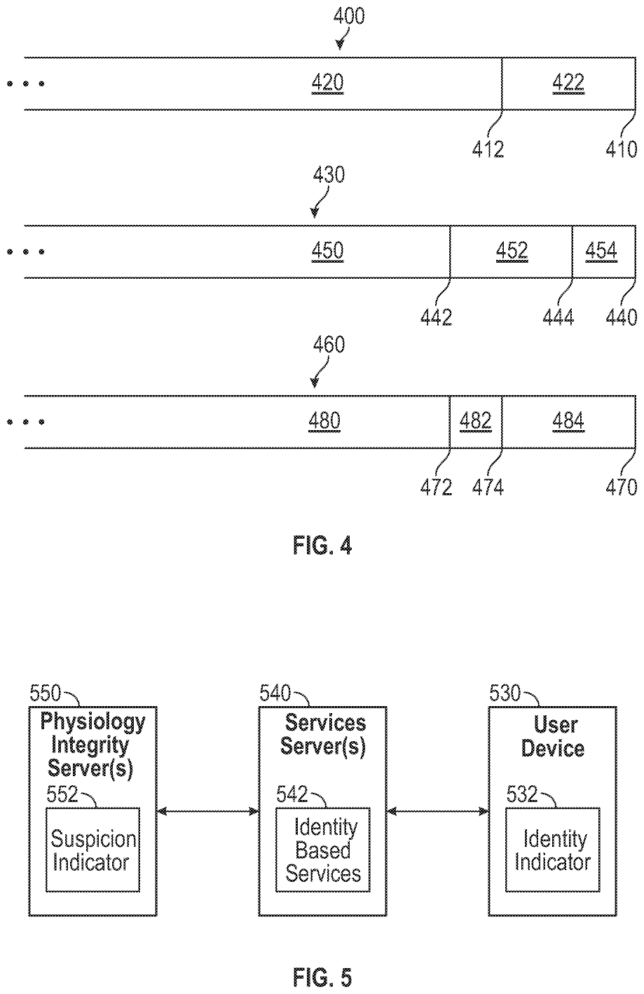

In various embodiments, the criteria may include statistical criteria and may refer to statistical or numerical models. For example, the computing device/system 310 may compute a mean of one or more temporal physiological data for the identity 320-320n over an earlier time period, and the physiology-based suspicion indicator 330 may be based on the degree to which temporal physiological data 320-320n over a recent time period deviates from the corresponding mean. In various embodiments, a smaller deviation may correspond to less suspicion, whereas a greater deviation may correspond to greater suspicion. Exemplary time periods and timelines are described below in connection with FIG. 4. In various embodiments, the statistical criteria may be based on other statistical measures, such as standard deviation or median, among other things. In various embodiments, the threshold value for indicating a suspicion regarding an identity may vary for different types of services. For example, for an authentication service which authenticates a login, the threshold value for indicating a suspicion regarding an entity may be configured such that suspicions are more easily triggered, whereas the threshold value for indicating a suspicion for an order placement service may be configured such that suspicions are less easily triggered. In various embodiments, the computing device/system 310 may include a “rules engine” which computes the physiology-based suspicion indicator for the identity 330, and the rules engine may include algorithms that learn and change as data is added and/or as data is removed. The rules engine may implement, for example, any of the criteria described herein for indicating a suspicion regarding an identity and/or may implement other criteria for indicating a suspicion regarding an identity.

In various embodiments, the computing device/system 310 may implement a trained machine learning model for the identity to generate the physiology-based suspicion indicator for the identity 330. The computing device/system 310 may implement various types of machine learning models, such as decision trees, support vector machines, and neural networks, among others. As persons skilled in the art will understand, training a machine learning model may involve using labeled training data to train the model. In various embodiments, deep learning techniques may be used such that the temporal physiological data 320-320n may be used without needing feature selection. In various embodiments, classical machine learning techniques may be used to perform feature selection to identify the most predictive temporal physiological data to use for the machine learning model. In such embodiments, the computing device/system 310 may only use a subset of the temporal physiological data for the identity 320-320n to generate the physiology-based suspicion indicator for the identity 330. Such and other embodiments are contemplated to be within the scope of the present disclosure. The computing device/system 310 may train a separate machine learning model for each identity.

According to some embodiments, the model for each identity includes a set of interdependencies between different features identified from the temporal physiological data and any other available data. The computing device/system 310 applies the corresponding model to the current available data to create the suspicion indicator. In this manner, the output of the models of each individual are based upon patient treatment data where such patient treatment data is not accessible through the same channels as other security information. For instance, facial recognition, login credentials, and security questions, are susceptible to theft or spoofing using different nefarious mechanisms. Aspects of the present disclosure can be particularly useful in light of the recognition that patient treatment data, and particularly temporal physiological data from an implantable medical device, may not be accessible through such mechanisms. Aspects of the present disclosure can also benefit from configuration of the computing device/system 310 such that the interface for receiving external requests for a suspicion identifier does not have access to (and is isolated from) the patient data and models.

In various embodiments, the computing device/system 310 may additionally use other data for the identity 322 to generate a physiology-based suspicion indicator for the identity 330. The other data 322 may not be physiological data. For example, the other data may include, without limitation, a signal from a physiological device of a person indicating that the physiological device is operational, location information for the person associated with the identity, and/or activity information for the person associated with the identity (e.g., sleep cycle, exercise routine, etc.), among other things. Such and other types of data may supplement the temporal physiological data 320-320n and are contemplated to be within the scope of the present disclosure. As mentioned above, statistical criteria, a rules engine, and/or machine learning may be used to generate a physiology-based suspicion indicator 330 based on one or more of the temporal physiological data 320-320n and the other data 322.

In various embodiments, the physiology-based suspicion indicator may be a value in a range, such as a value from zero through one-hundred, or a value in another range. In various embodiments, lower values may indicate less suspicion and higher values may indicate more suspicion. In various embodiments, lower values may indicate more suspicion and higher values may indicate less suspicion. In various embodiments, the physiology-based suspicion indicator may be a label rather than a value. In various embodiments, the labels may include, for example, “no suspicion”, “low suspicion”, “moderate suspicion”, and “high suspicion”, among others. In various embodiments, the labels may include, for example, “no identity integrity”, “low identity integrity”, “moderate identity integrity”, and “high identity integrity”, among others. Such values and labels are exemplary, and other embodiments which indicate a degree of suspicion are contemplated to be within the scope of the present disclosure (e.g., colors, images, etc.).

Various ways of generating the physiology-based suspicion indicator 330 are contemplated. In accordance with aspects of the present disclosure, the physiology-based suspicion indicator for an identity 330 is generated using temporal physiological data 320-320n (and optionally other data 322) which is stored remote from the person associated with the identity. Accordingly, in various embodiments, the physiology-based suspicion indicator 330 may be generated without using any data stored in a device on the person. In accordance with aspects of the present disclosure, the physiology-based suspicion indicator for an identity 330 may be generated using temporal physiological data 320-320n (and optionally other data 322) which was gathered before receiving the request to generate the physiology-based suspicion indicator 330. Accordingly, in various embodiments, the physiology-based suspicion indicator 330 may be generated without using any real-time query for physiological data. In accordance with aspects of the present disclosure, the physiology-based suspicion indicator 330 may be generated at regular and/or preset intervals. Accordingly, in various embodiments, the physiology-based suspicion indicator 330 may not be generated by request but may be provided upon request.

The examples shown and described in connection with FIG. 3 are illustrative, and variations are contemplated to be within the scope of the present disclosure. For example, a smaller number or a larger number of the temporal physiological data 320-320n may be used to determine the physiology-based suspicion indicator based on a desired level of scrutiny. A higher level of scrutiny may use a larger number of temporal physiological data, whereas a lower level of scrutiny may use a smaller number of temporal physiological data. In various embodiments, a physiology-based suspicion indicator may be provided for an account and/or for a group that is associated with multiple identities, rather than provided for a single identity. In various embodiments, the physiology-based suspicion indicator for the account or group may be computed based on the physiological data for one or more of the multiple identities. In various embodiments, the physiology-based suspicion indicator for the account or group may be computed based on the physiological data for all of the multiple identities. In various embodiments, a physiology-based suspicion indicator may be computed for each of the multiple identities, and the physiology-based suspicion indicator for the account or group may be based on the physiology-based suspicion indicator for each of the multiple identities. For example, the physiology-based suspicion indicator for the account or group may be an average, maximum, or minimum of the physiology-based suspicion indicator for each of the multiple identities, or may be another quantity. Such and other variations are contemplated to be within the scope of the present disclosure.

FIG. 4 shows a diagram of three exemplary timelines for the operations described in connection with FIG. 3. In a first exemplary timeline 400, the current time 410 coincides with the time when a physiology-based suspicion indicator is generated. A point of time 412 divides the timeline 400 into a reference time period 420 for computing a model, such as a statistical, numerical, rules-based, or machine learning model, among others, and an evaluation time period 422 for determining physiology-based suspicion. Physiological data gathered during the reference time period 420 may be used to generate the model, and physiological data gathered during evaluation time period 422 may be compared or applied to the model. As an example, physiological data gathered during the reference time period 420 may be used to compute a mean or median as a model. Physiological data gathered during the evaluation time period 422 may be used to compute a mean or median, which can be compared to the mean or median of the model. As mentioned above, a smaller deviation from the model may correspond to less suspicion, whereas a greater deviation from the model may correspond to greater suspicion. As another example, physiological data gathered during the reference time period 420 may be used to train a machine learning model, and physiological data gathered during the evaluation time period 422 may be used by the trained machine learning model to determine a physiology-based suspicion indicator. As an example, daily medication taken during the reference time period 420 and correlated heartrate over the reference time period 420 may be used to train a machine learning model, which may be applied at the time of a transaction to generate a physiology-based suspicion indicator based on medication taken and measured heartrate from a personal device during the evaluation time period. Such a physiology-based suspicion indicator may indicate, for example, an assurance level and/or a suspicion of fraud.

In various embodiments, the reference time period 420 may have a starting time point (not shown). In various embodiments, the point of time 412 may be twenty-four (24) hours before the current time, twelve (12) hours before the current time, six (6) hours before the current time, or another time period. In such embodiments, the length of time of the evaluation time period 422 may remain the same. In various embodiments, the point of time 412 may be fixed for a particular length of time and then updated regularly. For example, the point of time 412 may be fixed during the course of each day and may be advanced twenty-four hours once a day. In such embodiments, the length of time of the evaluation time period 422 may vary. In various embodiments, all of the physiological data during the evaluation time period 422 may be used for comparison to the model. In various embodiments, the evaluation time period 422 may be sub-divided into multiple time frames (not shown). The physiological data for each time frame may be separately compared to the model, and the comparisons of the time frames may be used in various ways to generate a physiology-based suspicion indicator.

In a second exemplary timeline 430, the current time 440 does not coincide with the time when a physiology-based suspicion indicator is generated. A point of time 442 divides the timeline 430 into a reference time period 450 for computing a model, such as a statistical, numerical, rules-based, or machine learning model, among others, and an evaluation time period 452 for determining physiology-based suspicion, and the physiology-based suspicion indicator is generated at a point of time 444. Physiological data gathered during the reference time period 450 may be used to generate the model, and physiological data gathered during evaluation time period 452 may be compared or applied to the model to generate the physiology-based suspicion indicator at point of time 444. The time between the point 444 when the physiology-based suspicion indicator is generated and the current time 440 may be referred to herein as a latency time period. For any request for a physiology-based suspicion indicator during the latency time period, the physiology-based suspicion indicator generated at point of time 444 is provided without generating a new physiology-based suspicion indicator.

In various embodiments, the reference time period 450 may have a starting time point (not shown). In various embodiments, the evaluation time period 452 may have a length of time that is twenty-four (24) hours after the reference time period, twelve (12) hours after the reference time period, six (6) hours after the reference time period, or another length of time. The time points 442, 444 may be advanced regularly such that the model and the physiology-based suspicion indicator are updated regularly. For example, the time points 442, 444 may be advanced every twenty-four (24) hours, every twelve (12) hours, every six (6) hours, or another time interval. In various embodiments, the evaluation time period 452 may be sub-divided into multiple time frames (not shown). The physiological data for each time frame may be separately compared to the model, and the comparisons of the time frames may be used in various ways to generate a physiology-based suspicion indicator.

In a third exemplary timeline 460, the current time 470 coincides with the time when a physiology-based suspicion indicator is generated. A point of time 472 indicates the end of a reference time period 480 for computing a model, such as a statistical, numerical, rules-based, or machine learning model, among others. The reference time period 480 may have a starting time point (not shown). A second point of time 474 indicates the beginning of the evaluation time period 484, and the evaluation time period 484 ends at the current time 470. Physiological data gathered during the reference time period 480 may be used to generate the model, and physiological data gathered during evaluation time period 484 may be compared or applied to the model to generate the physiology-based suspicion indicator at point of time 470. In the time period 482 between the reference time period 480 and the evaluation time period 484, the physiological data during that time period 482 is not used for generating either the model or the physiology-based suspicion indicator.

In various embodiments, the point of time 474 may be twenty-four (24) hours before the current time, twelve (12) hours before the current time, six (6) hours before the current time, or another time period. In such embodiments, the length of time of the evaluation time period 484 may remain the same. In various embodiments, the point of time 474 may be fixed for a particular length of time and then updated regularly. For example, the point of time 474 may be fixed during the course of each day and may be advanced twenty-four hours once a day. In such embodiments, the length of time of the evaluation time period 484 may vary. In various embodiments, all of the physiological data during the evaluation time period 484 may be used for comparison to the model. In various embodiments, the evaluation time period 484 may be sub-divided into multiple time frames (not shown). The physiological data for each time frame may be separately compared to the model, and the comparisons of the time frames may be used in various ways to generate a physiology-based suspicion indicator. In various embodiments, the point of time 472 may be advanced when the model is ready to be updated. The model updates may occur on a schedule and/or may occur on demand.

The examples shown and described in connection with FIG. 4 are illustrative, and variations are contemplated to be within the scope of the present disclosure. For example, aspects of the second exemplary timeline 430 and the third exemplary timeline 460 may be combined, such that the third exemplary timeline includes a latency time period. Such and other variations are contemplated to be within the scope of the present disclosure.

Referring now to FIG. 5, there is shown a diagram of an exemplary configuration for a services server to provide or not provide services to a user device based on a physiology-based suspicion indicator. FIG. 5 shows a user device/client device 530, a services system 540, and a physiology integrity system 550. Such user device/client devices and system 530-550 may be the user devices/client devices and systems described in connection with FIG. 1. Communications between the device/systems 530-550 may occur over one or more networks, which is not shown in FIG. 5 for clearer illustration.

In the illustrated embodiment, the user device 530 stores an identity indicator 532 and may request services from the services system 540. As mentioned above, the identity indicator 532 may be personal information (e.g., name, birthday, etc.), an e-mail address, a number (e.g., social security number, driver's license number, telephone number, etc.), a username, and/or an alphanumeric identifier, among other things. The user device 530 may communicate the identity indicator 532 to the services system 540. The services system 540 stores software that provides identity-based services 542. As mentioned above, the identity-based services 542 may include data access (e.g., e-mail access), data storage, product ordering, appointment booking, monetary services, electronic signature services, software as a service (SaaS), software as a medical device (SaMD), and/or other types of services. Based on the identity indicator 532 received from the user device 530, the services system 540 may request a physiology-based suspicion indicator 552 from the physiology integrity system 550. The physiology-based suspicion indicator for the identity 552 may be the physiology-based suspicion indicator 330 described in connection with FIG. 3. In various embodiments, the physiology-based suspicion indicator 552 may be generated by the physiology integrity system 550 when it receives the request from the services system 540. In various embodiments, the physiology-based suspicion indicator 552 may be generated according to preset time intervals or a preset schedule, and the physiology integrity system 550 may provide such pre-existing physiology-based suspicion indicator 552 when it receives the request from the services system 540. The services server 540 receives the physiology-based suspicion indicator 552 and, based on the indication provided by the physiology-based suspicion indicator 552, may decide to provide the requested services 542, may decline to provide the requested services 542, or may take another action, such as, for example, requesting further information from the user device 530 and/or providing an alert to the person associated with the identity, among other things. The examples shown and described in connection with FIG. 5 are illustrative, and variations are contemplated to be within the scope of the present disclosure.

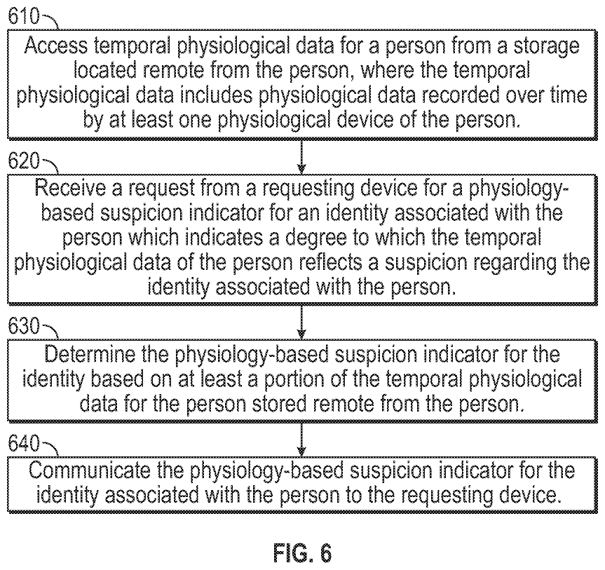

FIG. 6 shows a flow diagram of exemplary operations of a physiology integrity system, such as the physiology integrity system 150 FIG. 1 and/or the physiology integrity system 550 of FIG. 5. At block 610, the operation involves accessing temporal physiological data for a person from a storage located remote from the person. The storage may be located at the physiology integrity system, which is located remote from the person. The temporal physiological data may include physiological data recorded over time by one or more physiological devices of the person. As mentioned above, the physiological devices may be sensing devices which sense a physiological characteristic of a person, delivery devices which deliver a substance to a person, devices which stimulate a portion of a person's body, wearable devices, implanted devices, and/or carried devices, among other things.

At block 620, the operation involves receiving a request from a requesting device for a physiology-based suspicion indicator for an identity associated with the person. The requesting device may be, for example, a device of the services system described in connection with FIGS. 1 and 5. The physiology-based suspicion indicator may indicate a degree to which the temporal physiological data of the person reflects a suspicion regarding the identity associated with the person.

At block 630, the operation involves determining the physiology-based suspicion indicator for the identity based on a portion of or all of the temporal physiological data for the person which is stored remote from the person. Aspects of determining a physiology-based suspicion indicator are described above in connection with FIGS. 2 and 3. In various embodiments, the physiology-based suspicion indicator may be generated in response to the request from, e.g., the services system. In various embodiments, the physiology-based suspicion indicator may be pre-existing and may be accessed in response to the request from, e.g., the services system.

At block 640, the operation involves communicating the physiology-based suspicion indicator for the identity associated with the person to the requesting device, e.g., a device of the services system. As described above, the physiology-based suspicion indicator may be a value in a range, such as a value from zero through one-hundred, or a value in another range. In various embodiments, the physiology-based suspicion indicator may be a label rather than a value, such as, for example, “no suspicion”, “low suspicion”, “moderate suspicion”, and “high suspicion”, among others. Such examples of a physiology-based suspicion indicator are exemplary, and variations are contemplated to be within the scope of the present disclosure (e.g., colors, images, etc.). The examples shown and described in connection with FIG. 6 are illustrative, and variations are contemplated to be within the scope of the present disclosure.

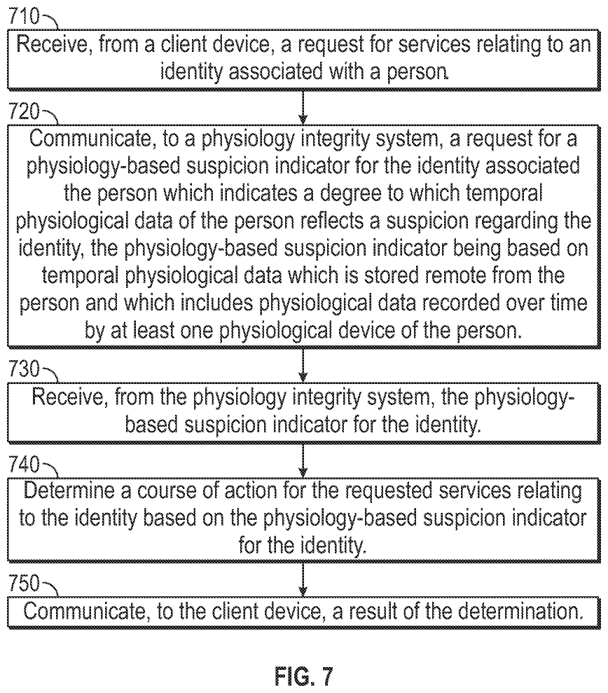

FIG. 7 shows a flow diagram of exemplary operations of a services system, such as the services system 140 FIG. 1 and/or the services system 540 of FIG. 5. At block 710, the operation involves receiving, from a user device/client device, a request for services relating to an identity associated with a person. As described above, the user device/client device may be any electronic device which can use electronic services of a system which provides services to user devices/client devices, including, without limitation, laptops, desktops, tablets, smartphones, smartwatches, dedicated devices for particular services (e.g., Ring® doorbell, automatic teller machine, etc.), dedicated devices for a particular person (e.g., insulin pump, pacemaker, etc.), and/or another type of electronic device. The services may be, for example, data access services (e.g., e-mail access), data storage services, product ordering services, appointment booking services, monetary services, electronic signature services, software as a service (SaaS), software as a medical device (SaMD), and/or other types of services.

At block 720, the operation involves communicating, to a physiology integrity system, a request for a physiology-based suspicion indicator for the identity associated the person. The physiology integrity system may be the physiology integrity system 150 of FIG. 1 and/or the physiology integrity system 550 of FIG. 5. The physiology-based suspicion indicator may indicate a degree to which temporal physiological data of the person reflects a suspicion regarding the identity. The physiology-based suspicion indicator may be based on temporal physiological data which is stored remote from the person, and which includes physiological data recorded over time by one or more physiological devices of the person. As mentioned above, the physiological devices may be sensing devices which sense a physiological characteristic of a person, delivery devices which deliver a substance to a person, devices which stimulate a portion of a person's body, wearable devices, implanted devices, and/or carried devices, among other things.

At block 730, the operation involves receiving, from the physiology integrity system, the physiology-based suspicion indicator for the identity. As described above, the physiology-based suspicion indicator may be a value in a range, such as a value from zero through one-hundred, or a value in another range. In various embodiments, the physiology-based suspicion indicator may be a label rather than a value, such as, for example, “no suspicion”, “low suspicion”, “moderate suspicion”, and “high suspicion”, among others. Such examples of a physiology-based suspicion indicator are exemplary, and variations are contemplated to be within the scope of the present disclosure (e.g., colors, images, etc.).

At block 740, the operation involves determining a course of action for the requested services relating to the identity based on the physiology-based suspicion indicator for the identity. For example, based on the indication provided by the physiology-based suspicion indicator, the course of action may provide the requested services, decline to provide the requested services, and/or other courses of action, such as requesting further information or providing an alert to the person associated with the identity, among other things.

At block 750, the operation involves communicating, to the user device client device, a result of the determination. For example, if the determined course of action is to provide the requested services, a screen relating to the requested service may be communicated to the client device/user device. If the determined course of action is to not provide the requested services, a screen relating to the denied service may be communicated to the client device/user device. Other courses of actions may be communicated as appropriate. The examples shown and described in connection with FIG. 7 are illustrative, and variations are contemplated to be within the scope of the present disclosure.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

The systems, devices, and/or servers described herein may utilize one or more processors to receive various information and transform the received information to generate an output. The processors may include any type of computing device, computational circuit, or any type of controller or processing circuit capable of executing a series of instructions that are stored in a memory. The processor may include multiple processors and/or multicore central processing units (CPUs) and may include any type of device, such as a microprocessor, graphics processing unit (GPU), digital signal processor, microcontroller, programmable logic device (PLD), field programmable gate array (FPGA), or the like. The processor may also include a memory to store data and/or instructions that, when executed by the one or more processors, causes the one or more processors to perform one or more methods and/or algorithms.

Any of the herein described operations, methods, programs, algorithms, or codes may be converted to, or expressed in, a programming language or computer program embodied on a computer, processor, or machine-readable medium. The terms “programming language” and “computer program,” as used herein, each include any language used to specify instructions to a computer or processor, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. To the extent consistent, any or all of the aspects detailed herein may be used in conjunction with any or all of the other aspects detailed herein. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A system comprising:
at least one processor; and
one or more memories storing instructions which, when executed by the at least one processor, cause the system to:
access temporal physiological data for a person from a storage located remote from the person, the temporal physiological data including physiological data recorded over time by at least one physiological device of the person;
receive a request from a requesting device for a physiology-based suspicion indicator for an identity associated with the person which indicates a degree to which the temporal physiological data of the person reflects a suspicion regarding the identity associated with the person;
determine the physiology-based suspicion indicator for the identity based on at least a portion of the temporal physiological data for the person stored remote from the person; and
communicate the physiology-based suspicion indicator for the identity associated with the person to the requesting device.

2. The system of claim 1, wherein the physiology-based suspicion indicator for the identity is determined based on only data gathered prior to the request from the requesting device.

3. The system of claim 1, wherein the physiology-based suspicion indicator for the identity is determined based on only data stored remote from the person associated with the identity.

4. The system of claim 1, wherein the at least one physiological device includes a treatment delivery device,
wherein the physiological data recorded over time includes treatment measurements recorded over time by the treatment delivery device, and
wherein the physiology-based suspicion indicator for the identity indicates a greater degree of suspicion when the treatment measurements recorded over time indicate non-recommended treatment delivery to the person.

5. The system of claim 1, wherein the temporal physiological data for the person is accessed from among a plurality of stored data for the person,
wherein the instructions, when executed by the at least one processor, further cause the system to:
analyze degrees of correlation between the plurality of stored data and a plurality of physiology-based suspicion indicators, and
select the temporal physiological data from among the plurality of stored data based on the degrees of correlation.

6. The system of claim 1, wherein the physiology-based suspicion indicator for the identity is determined based on applying at least one of a rules engine or a trained machine learning model to the at least the portion of the temporal physiological data for the person.

7. The system of claim 6, wherein the trained machine learning model is trained based on data specific to the person and is customized to the person.

8. A processor-implemented method comprising:
accessing temporal physiological data for a person from a storage located remote from the person, the temporal physiological data including physiological data recorded over time by at least one physiological device of the person;
receiving a request from a requesting device for a physiology-based suspicion indicator for an identity associated with the person which indicates a degree to which the temporal physiological data of the person reflects a suspicion regarding the identity associated with the person;
determining the physiology-based suspicion indicator for the identity based on at least a portion of the temporal physiological data for the person stored remote from the person; and
communicating the physiology-based suspicion indicator for the identity associated with the person to the requesting device.

9. The processor-implemented method of claim 8, wherein determining the physiology-based suspicion indicator for the identity includes determining the physiology-based suspicion indicator for the identity based on only data gathered prior to the request from the requesting device.

10. The processor-implemented method of claim 8, wherein determining the physiology-based suspicion indicator for the identity includes determining the physiology-based suspicion indicator for the identity based on only data stored remote from the person associated with the identity.

11. The processor-implemented method of claim 8, wherein the at least one physiological device includes a treatment delivery device,
wherein the physiological data recorded over time includes treatment measurements recorded over time by the treatment delivery device, and
wherein the physiology-based suspicion indicator for the identity indicates a greater degree of suspicion when the treatment measurements recorded over time indicate non-recommended treatment delivery to the person.

12. The processor-implemented method of claim 8, wherein the temporal physiological data for the person is accessed from among a plurality of stored data for the person,
the processor-implemented method further comprising:
analyzing degrees of correlation between the plurality of stored data and a plurality of physiology-based suspicion indicators, and
selecting the temporal physiological data from among the plurality of stored data based on the degrees of correlation.

13. The processor-implemented method of claim 8, wherein determining the physiology-based suspicion indicator for the identity includes determining the physiology-based suspicion indicator for the identity based on applying at least one of a rules engine or a trained machine learning model to the at least the portion of the temporal physiological data for the person.

14. The processor-implemented method of claim 13, wherein the trained machine learning model is trained based on data specific to the person and is customized to the person.

15. A non-transitory processor-readable medium storing instructions which, when executed by at least one processor of a system, cause the system to:
access temporal physiological data for a person from a storage located remote from the person, the temporal physiological data including physiological data recorded over time by at least one physiological device of the person;
receive a request from a requesting device for a physiology-based suspicion indicator for an identity associated with the person which indicates a degree to which the temporal physiological data of the person reflects a suspicion regarding the identity associated with the person;
determine the physiology-based suspicion indicator for the identity based on the temporal physiological data for the person stored remote from the person; and
communicate the physiology-based suspicion indicator for the identity associated with the person to the requesting device.

16. The non-transitory processor-readable medium of claim 15, wherein the physiology-based suspicion indicator for the identity is determined based on only data gathered prior to the request from the requesting device.

17. The non-transitory processor-readable medium of claim 15, wherein the physiology-based suspicion indicator for the identity is determined based on only data stored remote from the person associated with the identity.

18. The non-transitory processor-readable medium of claim 15, wherein the at least one physiological device includes a treatment delivery device,
wherein the physiological data recorded over time includes treatment measurements recorded over time by the treatment delivery device, and
wherein the physiology-based suspicion indicator for the identity indicates a greater degree of suspicion when the treatment measurements recorded over time indicate non-recommended treatment delivery to the person.

19. The non-transitory processor-readable medium of claim 15, wherein the temporal physiological data for the person is accessed from among a plurality of stored data for the person,
wherein the instructions, when executed by the at least one processor, further cause the system to:
analyze degrees of correlation between the plurality of stored data and a plurality of physiology-based suspicion indicators, and
select the temporal physiological data from among the plurality of stored data based on the degrees of correlation.

20. The non-transitory processor-readable medium of claim 15, wherein the physiology-based suspicion indicator for the identity is determined based on applying at least one of a rules engine or a trained machine learning model to the at least the portion of the temporal physiological data for the person.
